(19) United States
(12) Patent Application Publication
(10) Pub. No.: US 2024/0410846 A1
(43) Pub. Date: Dec. 12, 2024

(54) METHODS FOR INCREASING THE ACCURACY OF A HUMIDITY SENSOR

(71) Applicant: NANOSCENT LTD., Misghav (IL)

(72) Inventors: Sima Simha ISRAEL, Atsmon Segev (IL); Roie SHLOMOVITZ, Kiryat Tivon (IL); Samah ABU ELHIJA, Tamra (IL); Maria GOLDSTEIN, Nahariyya (IL)

(73) Assignee: NANOSCENT LTD., Misghav (IL)

(21) Appl. No.: 18/738,338

(22) Filed: Jun. 10, 2024

Related U.S. Application Data
(60) Provisional application No. 63/472,301, filed on Jun. 11, 2023.

(51) Int. Cl. G01N 25/56; G01N 33/00

(52) U.S. Cl. CPC G01N 25/56; G01N 33/0006

(57) ABSTRACT

A method for increasing the accuracy of a humidity sensor is disclosed. The method comprises: receiving one or more first sets of humidity measurements from a humidity sensor over a period of time, when the humidity sensor is exposed a to a first gas sample having a known humidity level; determining a calibrating humidity measurement from the first sets of humidity measurements; receiving one or more second sets of humidity measurements from the humidity sensor over a period of time, when the humidity sensor is exposed to a second gas sample having an unknown humidity level; and for each second set, subtracting the calibrating humidity measurement from the humidity measurements.

### CROSS REFERENCE TO RELATED APPLICATIONS

This application claims the benefit of priority under 35 U.S.C. § 119 (e) of U.S. Provisional Patent Application No. 63/472,301, filed 11 Jun. 2023, entitled “METHODS FOR INCREASING THE ACCURACY OF A HUMIDITY SENSOR”. The contents of the above application is all incorporated by reference as if fully set forth herein in its entirety.

### FIELD OF THE INVENTION

The present invention relates generally to humidity sensors. More specifically, the present invention relates to methods for increasing the accuracy of a humidity sensor.

### BACKGROUND OF THE INVENTION

A humidity sensor, also known as a Hygrometer is a device that detects and measures water vapor. Humidity sensors can measure the absolute humidity in the air, in concentration from total molecules, and the like. Alternatively, Humidity (in water vapor pressure) sensors can measure the ‘relative humidity’ (RH) which is defined as the ratio of the partial pressure of water vapor also known as ‘absolute humidity’ (AH) in air to the saturation vapor pressure (SVP) of water at the same temperature, usually expressed as a percentage, using for example, equations 1 and 2.

$$RH = \frac{AH}{SVP} \times 100 \% \qquad ( 1 )$$

$${C}_{H 2 O} [ {PPM}_{v} ] = \frac{AH}{{P}_{sys} - AH} * 1 {0}^{6} \qquad ( 2 )$$

Wherein, C_{H2O} is the concentration of water in PPMᵥ units, and P_{sys} is the total pressure in the system. In low AH pressure the AH in the denominator can be neglected.

Modern hygrometers measure humidity using various physical properties. For example, in capacitive hygrometers, the effect of humidity on the dielectric constant of a polymer or metal oxide material is measured. With calibration, these sensors have an accuracy of ±2% RH in the range 5-95% RH.

In resistive hygrometers, the change in electrical resistance of a material due to humidity is measured. Typical materials are salts and conductive polymers. Resistive sensors are less sensitive than capacitive sensors.

Thermal hygrometers, measure the change in thermal conductivity of air due to humidity. These sensors measure absolute humidity rather than relative humidity.

An optical hygrometer measures the absorption of light by water in the air. A light emitter and a light detector are arranged with a volume of air between them. The attenuation of the light, as seen by the detector, indicates the humidity, according to the Beer-Lambert. Types include the Lyman-alpha hygrometer (using Lyman-alpha light emitted by hydrogen), the krypton hygrometer (using 123.58 nm light emitted by krypton), and the differential absorption hygrometer (using light emitted by two lasers operating at different wavelengths, one absorbed by humidity and the other not).

However, most of these hygrometers are sensitive to changes in temperature and pressure. Furthermore, a hygrometer capable of measuring less than 1% RH is very costly and complicated to operate, for example, the gravimetric hygrometer which extracts the water from the air.

Accordingly, there is a need for simple methods allowing to use commercial hygrometer, such as, capacitive hygrometers or resistive hygrometers and improve their accuracy, regardless of the temperature and/or the pressure at which the measurement was taken.

### SUMMARY OF THE INVENTION

Some aspects of the invention may be related a method for increasing the accuracy of a humidity sensor, comprising: receiving one or more first sets of humidity measurements from a humidity sensor over a period of time, when the humidity sensor is exposed a to a first gas sample having a known humidity level; determining a calibrating humidity measurement from the first sets of humidity measurements; receiving one or more second sets of humidity measurements from the humidity sensor over a period of time, when the humidity sensor is exposed to a second gas sample having an unknown humidity level; and for each second set, subtracting the calibrating humidity measurement from the humidity measurements.

In some embodiments, the humidity measurements are taken at a temperature selected based on the required sensitivity. In some embodiments, the humidity measurements are taken at a temperature lower than 5° C. In some embodiments, the humidity measurements are given at relative humidity (RH) units and the method further comprises: conducting a temperature calibration process comprising: receiving first calibration RH measurements from the humidity sensor, taken at various temperatures, using a sample with known RH; extracting at least one first calibration parameter from the calibration RH measurements; and determining a calibration temperature, based on the various temperatures; and calibrating one or more second sets of RH measurements using the at least one first calibration parameter and the calibration temperature. In some embodiments, the calibration temperature is selected from, an average temperature of the various temperatures, a median temperature of the various temperatures, a first temperature from the various temperatures, and a working temperature at which the second gas sample is provided. In some embodiments, the at least one first calibration parameter is a gradient of a linear relationship between the calibration RH measurements and the various temperatures.

In some embodiments, the humidity measurements are given at relative humidity (RH) units and the method further comprises: conducting a pressure calibration process comprising: receiving second calibration humidity measurements from the humidity sensor, taken at various pressure levels, using a sample with known RH; extracting at least one second calibration parameter from the second calibration RH measurements; and determining a calibration pressure, based on the various pressure levels; and calibrating the one or more third sets of RH measurements using the at least one second calibration parameter and the calibration pressure. In some embodiments, the calibration pressure is selected from, an average pressure level of the various pressure levels, a median pressure level of the various pressure levels, a first pressure level from the various pressure levels, and a working pressure level at which the second gas sample is provided. In some embodiments, the at least one second calibration parameter is a gradient of a linear relationship between the calibration RH measurements and the various pressure levels.

In some embodiments, the method further comprises displaying the calibrated one or more second sets of humidity measurements.

Some additional aspects of the invention are related to a method for increasing the accuracy of measurements of a humidity sensor, comprising: receiving relative humidity (RH) measurements from a humidity sensor, taken at various temperatures, using a sample with known RH; extracting at least one calibration parameter from the RH measurements; determining a calibration temperature, based on the various temperatures; and calibrating one or more additional sets of RH measurements using the at least one calibration parameter and the calibration temperature.

In some embodiments, the calibration temperature is selected from, an average temperature of the various temperatures, a median temperature of the various temperatures, a first temperature from the various temperatures, and a working temperature at which the second gas sample is provided. In some embodiments, the at least one calibration parameter is a gradient of a linear relationship between the RH measurements and the various temperatures.

Some additional aspects of the invention are related to a method for increasing the accuracy of measurements from a humidity sensor, comprising: receiving relative humidity (RH) measurements from a humidity sensor, taken at various pressure levels, using a sample with known RH; extracting at least one calibration parameter from the RH measurements; determining a calibration pressure, based on the various pressure levels; and calibrating the one or more additional sets of RH measurements using the at least one calibration parameter and the calibration pressure.

In some embodiments, the calibration pressure is selected from, an average pressure level of the various pressure levels, a median pressure level of the various pressure levels, a first pressure level from the various pressure levels, and a working pressure level at which the second gas sample is provided. In some embodiments, the at least one second calibration parameter is a gradient of a linear relationship between the calibration RH measurements and the various pressure levels.

Some additional aspects of the invention are related to a system for increasing the accuracy of humidity measurements, comprising: a humidity sensor; and a controller configured to: receive one or more first sets of humidity measurements from the humidity sensor over a period of time, when the humidity sensor is exposed to a first gas sample having a known humidity level; determine a calibrating measurement from the first sets of humidity measurements; receive one or more second sets of humidity measurements from the humidity sensor over a period of time, when the humidity sensor is exposed a to a second gas sample having an unknown humidity level; and for each second set, subtracting the calibrating measurement from the humidity measurements.

Some additional aspects of the invention are related to a system for increasing the accuracy of humidity measurements, comprising: a humidity sensor; a temperature sensor; and a controller configured to: receive relative humidity (RH) measurements from the humidity sensor, taken at various temperatures, using a sample with known RH, wherein the temperatures are simultaneously measured by the temperature sensor; extract at least one calibration parameter from the RH measurements; determine a calibration temperature, based on the various temperatures; and calibrate one or more additional sets of RH measurements using the at least one calibration parameter and the calibration temperature.

Some additional aspects of the invention are related to system for increasing the accuracy of humidity measurements, comprising: a humidity sensor; a pressure sensor; and a controller configured to: receive relative humidity (RH) measurements from the humidity sensor, taken at various pressure levels, using a sample with known RH, wherein the pressure levels are simultaneously measured by the pressure sensor; extract at least one calibration parameter from the RH measurements; determine a calibration pressure, based on the various pressure levels; and calibrate one or more additional sets of RH measurements using the at least one calibration parameter and the calibration pressure.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a block diagram of a system for increasing the accuracy of humidity measurements according to some embodiments of the invention;

FIG. 1B is a block diagram, depicting a computing device which may be included in a system for increasing the accuracy of humidity measurements according to some embodiments of the invention;

FIG. 2A is a flowchart of a method for increasing the accuracy of a humidity sensor according to some embodiments of the invention;

FIG. 2B includes graphs showing humidity measurements according to some embodiments of the invention;

FIG. 2C includes a graph showing the sensitivity of the humidity measurements to exposure at various temperatures, according to some embodiments of the invention;

FIG. 3A is a flowchart of another method for increasing the accuracy of measurements of a humidity sensor, under various temperatures according to some embodiments of the invention;

FIG. 3B; shows a graph of humidity measurements vs. temperature according to some embodiment of the invention;

FIG. 3C shows a graph of RH measurements, temperature measurements and RH calibration, according to some embodiments of the invention; and

FIG. 4 is a flowchart of another method for increasing the accuracy of measurements of a humidity sensor, under various pressure levels according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Embodiments of the present invention disclose a method of increasing the accuracy of commercial humidity sensors (i.e., hygrometers), as to allow them to accurately measure less than 5% RH. Some additional embodiments include calibration methods that can overcome the sensitivity of the RH measurements to variations in temperature and pressure levels.

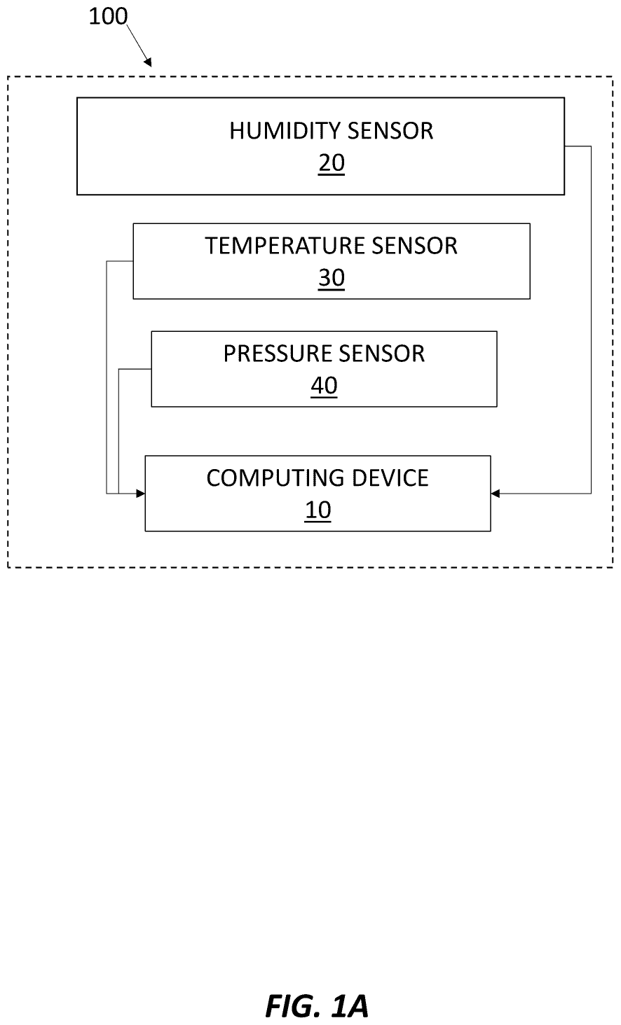

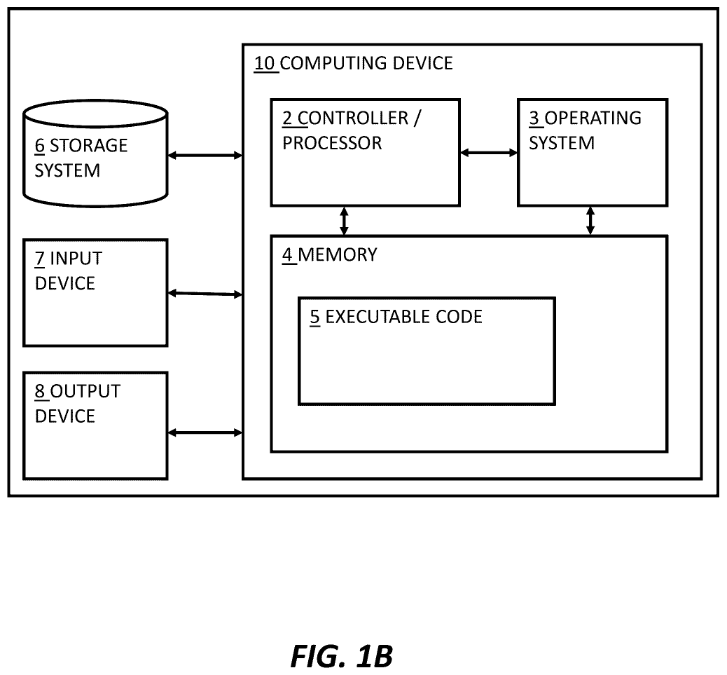

Reference is now made to FIG. 1A which is a block diagram of a system for increasing the accuracy of measurements of a humidity sensor according to some embodiments of the invention. A sensing system 100 according to embodiments of the invention may include a humidity sensor 20. Humidity sensor 20 may be any commercial or known humidity sensors, for example, a capacitive hygrometer, a resistive hygrometer, and the like. In some embodiments, humidity sensor 20 may be configured to measure the RH, the AH or both.

In some embodiments, system 100 may further include a temperature sensor 30 located inside in proximity to humidity sensor 20, as to measure the temperature of a gas sample to which humidity sensor 20 is exposed. For example, temperature sensor 30 may be packed in the same housing as humidity sensor 20, attached to humidity sensor 20, or located in the pathway of the gas sample prior to being exposed to humidity sensor 20, and the like.

In some embodiments, system 100 may further include a pressure sensor 40 located inside in proximity to humidity sensor 20, as to measure the pressure level of the gas sample to which humidity sensor 20 is exposed. For example, pressure sensor 40 (e.g., a barometer) may be packed in the same housing as humidity sensor 20, attached to humidity sensor 20, or located in the pathway of the gas sample prior to being exposed to humidity sensor 20, and the like.

Sensing system 100 may further include a computing device 10 discussed in detail with respect to FIG. 1B.

Reference is now made to FIG. 1B, which is a block diagram depicting a computing device, which may be included within an embodiment of a system for increasing the accuracy of measurements of a humidity sensor, according to some embodiments.

Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 10, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may in-situ increase the accuracy of measurements of a humidity sensor as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Mathematical correlation to be used for increasing the accuracy of a humidity sensor may be stored in storage system 6 and may be loaded from storage system 6 into memory 4 where it may be processed by processor or controller 2. In some embodiments, some of the components shown in FIG. 1B may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 10 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 10 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

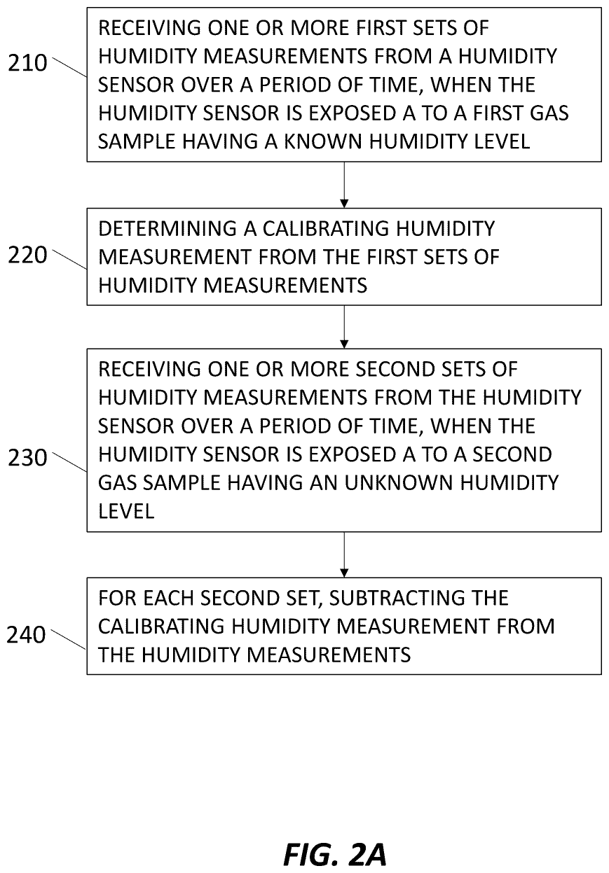

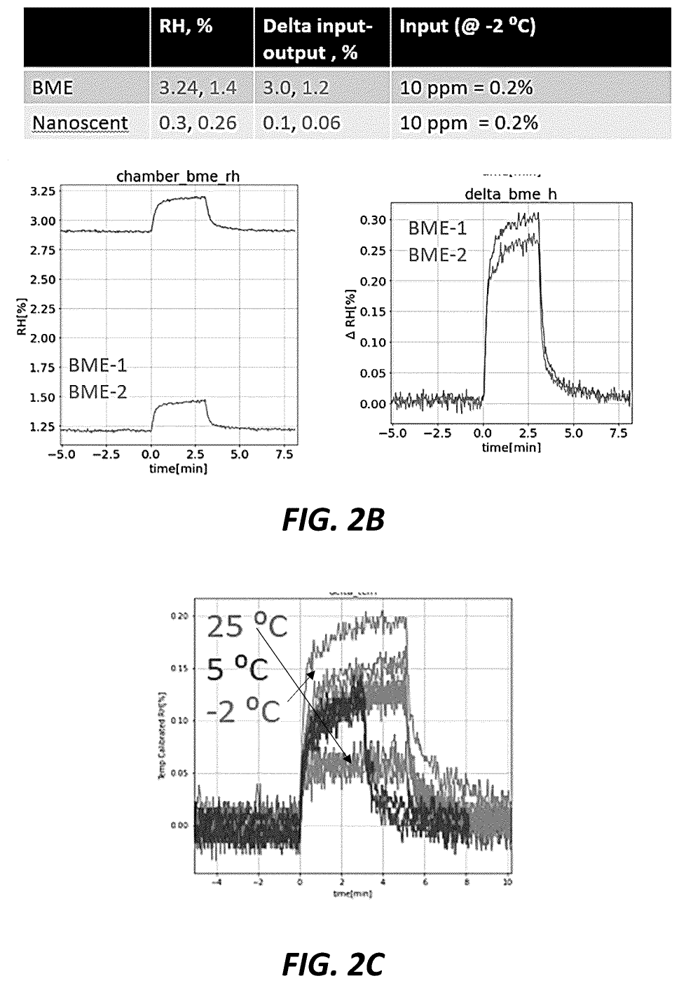

Reference is now made to FIG. 2A which is a flowchart of a method for increasing the accuracy of a humidity sensor according to some embodiments of the invention. The method of FIG. 2A may be conducted by controller 2 of computing device 100 included in system 100.

In step 210, one or more first sets of humidity measurements may be received from a humidity sensor over a period of time, when the humidity sensor is exposed to a first gas sample having a known humidity level. In some embodiments, computing device 10 may receive one or more first sets of humidity measurements from humidity sensor 20. A nonlimiting example for such measurements is given in the left graph of FIG. 2B at which in the first and last 5 minutes sensor 20 was exposed to dry nitrogen having no more than AR of 0.1 ppm at a constant temperature of −2° C. and atmospheric pressure. In yet another nonlimiting example, in the first and last 5 minutes sensor 20 was exposed to nitrogen having no more than AR of 10 ppm at a constant temperature of −2° C. and atmospheric pressure.

As clearly shown in the left graph of FIG. 2B, although exposed to the same sample, at the same temperature and pressure, each measurement yields a different RH value. The measured maxima values, 1.5% RH and 3.2% RH are much larger than the maximal 0.2% RH of the exposed sample.

In step 220, a calibrating humidity measurement may be determined from the first sets of humidity measurements. In some embodiments, computing device 10 may determine a calibrating humidity measurement for each one of the received sets. For example, the calibrating humidity measurement may be the average humidity measurement measured during the exposure of sensor 20 to the sample having a known humidity level (e.g., 0.002% RH). In yet another example, the calibrating humidity measurement may be the last measurement, a measurement received after a predetermined time (e.g., 1 min, 2, min. 3 min. etc.,) and the like. In the nonlimiting example of FIG. 2B, the calibrating humidity measurement is the average humidity measurement of all the measurements received during the last 3 minutes.

In step 230, one or more second sets of humidity measurements may be received from the humidity sensor over a period of time, when the humidity sensor is exposed to a second gas sample having an unknown humidity level. In some embodiments, humidity sensor 20 may be exposed to a sample having an unknown humidity. For example, as shown in the left graph of FIG. 2B, the sensor was exposed, after 5 minutes to a sample having RH of 0.2% for 3 minutes and then flashed with dry gas (e.g., Nitrogen). A shown both graphs, in both measurements showed a rise in the RH however provided two very different measurements, 1.5% and 3.2% RH, which are one order of magnitude larger than the real RH of 0.2%.

In step 240, the calibrating humidity measurement may be subtracted from the humidity measurements, of each set. For example, as shown in the right graph of FIG. 2A, which shows the subtracted measurements, the RH of the dry air is close to zero and the RH of the sample is between 0.2 to 0.3% RH, which is much closer to the actual RH of 0.2%.

Accordingly, the method of FIG. 2A improves the accuracy of the measurement even at very low RH percentages, e.g., below 2%, 1.5%, 1%, 0.8%, 0.5%, 0.4%, 0.3%, 0.2%, 0.1%, 0.05%, 0.01% and any value in between.

In some embodiments, in order to further improve the accuracy of the sensor, the measurements should be taken under controlled temperature. In some embodiments, the humidity measurements may be taken at a temperature selected based on the required sensitivity. For example, as shown in the graphs in FIG. 2C the lower the temperature the higher is the signal measured by the sensor. The inventors surprisingly found, that in order to measure low RH percentages, e.g., below 1%, the maximum temperature should be 5° C.

In some embodiments, when the humidity is measured in RH, additional calibration may be required in order to ensure receiving accurate RH measurements.

RH is known to vary with the variation of temperature and/or pressure. Unlike AH which measures the amount of water molecules, RH is the ratio between AH and SVP. The SVP is sensitive to variations of temperature, while C_{H2O} is sensitive to variations in pressure.

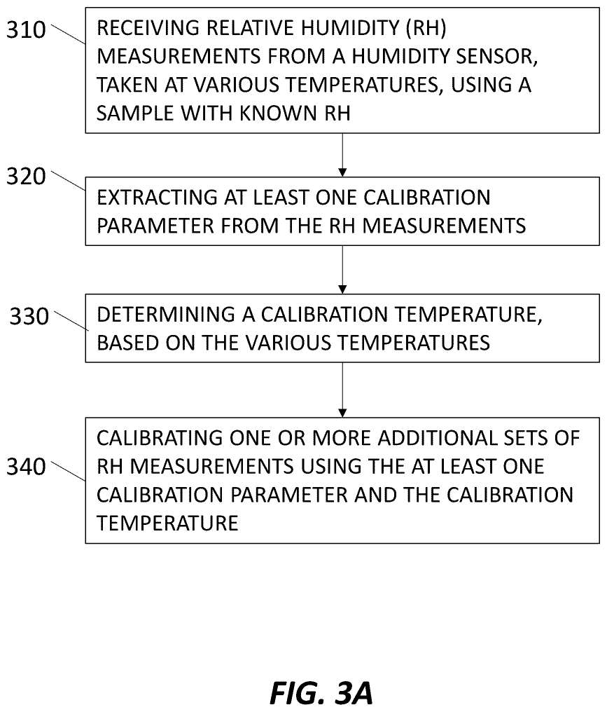

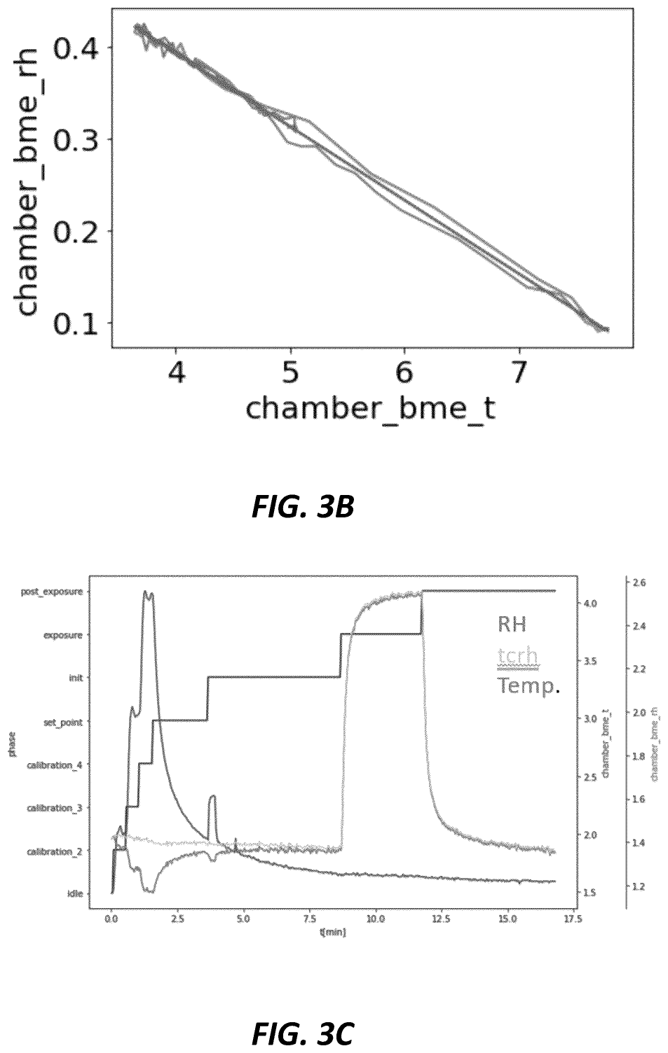

Reference is now made to FIG. 3A which is a method for increasing the accuracy of measurements of a humidity sensor according to some embodiments of the invention. Performing the method of FIG. 3A may allow to decrease the influence of temperature variations of the measurement of RH. The method of FIG. 3A may be performed by system 100, using both humidity sensor 20 and temperature sensors 30, under the supervision of computing device 10.

In step 310, relative humidity (RH) measurements may be received from a humidity sensor, taken at various temperatures, using a sample with known RH. In some embodiments, sensor 20 may be exposed to a gas sample having known humidity (e.g., dray air with less than 0.001% RH). The RH measurements may be raw RH measurements, as received directly from sensor 20 and shown in the left graph of FIG. 2A. In some embodiments, the RH measurements may be subtracted measurements, resulted from conducting the method of FIG. 2A. Therefore, the RH measurements may be the measurements of step 240, shown in the right graph of FIG. 2B. Both the raw or subtracted measurements may be taken at various temperatures. In a nonlimiting example, RH measurements of dry air were taken at temperatures varying from 3 to 9°° C., as shown in FIG. 3B. The RH measurement in each temperature may be an average temperature taken, for example, over a period of time (e.g., 5 minutes) at the same temperature.

In step 320, at least one calibration parameter may be extracted from the RH measurements. In some embodiments, computing device 10 may analyze the RH measurements taken at the temperatures to extract the parameter. For example, the at least one calibration parameter may be a gradient of a linear relationship between the RH measurements and the various temperatures, as illustrated in FIG. 3A and derived from equation (3), showing the dependency of the RH on the temperature.

$$RH = a ( T ) + b \qquad ( 3 )$$

Where, ‘T’ is the temperature and ‘a’ the gradient, and ‘b’ is an additional parameter.

In step 330, a calibration temperature may be determined based on the various temperatures. In some embodiments, the calibration temperature is selected from, an average temperature of the various temperatures, a median temperature of the various temperatures, a first temperature from the various temperatures, and a working temperature at which the second gas sample is provided (as shown in FIG. 3C) and the like.

In step 340, one or more additional sets of RH measurements may be calibrated using the at least one calibration parameter and the calibration temperature.

In some embodiments, a calibration function may be derived based on the calibration temperature and the at least one calibration parameter, for example, using equation 4.

$${RH}_{CLB} ( t ) = RH ( t ) - a ( T ( t ) - {T}_{0} ) \qquad ( 4 )$$

Where, RH_{CLB}(t) are the time dependent calibrated RH measurements, RH(t) the time dependent RH measurements, T(t) the time dependent temperature measurements, T₀ the calibration temperature and ‘a’ the calibration parameter.

A nonlimiting example of such calibration is shown in FIG. 3C. The raw RH(t) is presented by a dark grey line and the calibrated RH_{CLB}(t) by a light grey line. The temperature measurements are denoted by a darker grey line. As shown in FIG. 3C the RH_{CLB}(t) is not affected from the changes in the temperature.

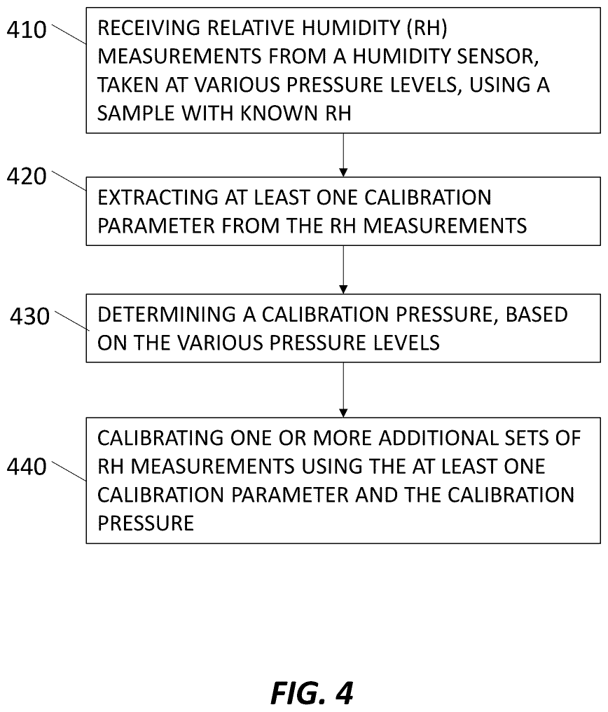

Similar effects on the RH measurements may derived from changes in pressure. Accordingly, reference is now made to FIG. 4 which is a method for increasing the accuracy of measurements of a humidity sensor according to some embodiments of the invention. Performing the method of FIG. 4 may allow to decrease the influence of pressure variation on the measurement of RH. The method of FIG. 4 may be performed by system 100, using both humidity sensor 20 and pressure sensors 40, under the supervision of computing device 10.

In step 410, relative humidity (RH) measurements may be received from a humidity sensor, taken at various pressure levels, using a sample with known RH. In some embodiments, the RH measurements may be raw RH measurements, as received directly from sensor 20 and shown in the left graph of FIG. 2A. In some embodiments, the RH measurements may be subtracted measurements, resulting from conducting the method of FIG. 2A. Therefore, the RH measurements may be the measurements of step 240, shown in the right graph of FIG. 2B. Both the raw or subtracted measurements may be taken at various pressure levels.

In step 420, at least one calibration parameter may be extracted from the RH measurements. In some embodiments, computing device 10 may analyze the RH measurements taken at the various pressure levels to extract the parameter. For example, the at least one calibration parameter may be a gradient of a linear relationship between the RH measurements and the various pressure levels, as derived from equation (5), showing the dependency of the RH on the temperature.

$$RH = c ( P ) + d \qquad ( 5 )$$

Where, ‘P’ is the temperature and ‘c’ the gradient.

In step 330, a calibration pressure may be determined based on the various pressure levels. In some embodiments, the calibration pressure is selected from, an average pressure level of the various pressure levels, a median pressure level of the various pressure levels, a first pressure level from the various pressure levels, and a working pressure level at which the gas sample is provided and the like.

In step 440, one or more additional sets of RH measurements may be calibrated using the at least one calibration parameter and the calibration pressure.

In some embodiments, a calibration function may be derived based on the calibration pressure and the at least one calibration parameter, for example, using equation 6.

$${RH}_{CLBP} ( t ) = R H ( t ) - c ( P ( t ) - {P}_{0} ) \qquad ( 6 )$$

Where, RH_{CLBP}(t) are the time dependent calibrated RH measurements, RH(t) the time dependent RH measurements, P(t) the time dependent pressure level measurements, Po the calibration pressure and ‘c’ the calibration parameter.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method for increasing the accuracy of a humidity sensor, comprising:
receiving one or more first sets of humidity measurements from a humidity sensor over a period of time, when the humidity sensor is exposed a to a first gas sample having a known humidity level;
determining a calibrating humidity measurement from the first sets of humidity measurements;
receiving one or more second sets of humidity measurements from the humidity sensor over a period of time, when the humidity sensor is exposed to a second gas sample having an unknown humidity level; and
for each second set, subtracting the calibrating humidity measurement from the humidity measurements.

2. The method according to claim 1, wherein the humidity measurements are taken at a temperature selected based on the required sensitivity.

3. The method according to claim 1, wherein the humidity measurements are taken at a temperature lower than 5° C.

4. The method according to claim 1, wherein the humidity measurements are given at relative humidity (RH) units and the method further comprises:
conducting a temperature calibration process comprising:
receiving first calibration RH measurements from the humidity sensor, taken at various temperatures, using a sample with known RH;
extracting at least one first calibration parameter from the calibration RH measurements; and
determining a calibration temperature, based on the various temperatures; and
calibrating one or more second sets of RH measurements using the at least one first calibration parameter and the calibration temperature.

5. The method of claim 4, wherein the calibration temperature is selected from, an average temperature of the various temperatures, a median temperature of the various temperatures, a first temperature from the various temperatures, and a working temperature at which the second gas sample is provided.

6. The method of claim 4, wherein the at least one first calibration parameter is a gradient of a linear relationship between the calibration RH measurements and the various temperatures.

7. The method according to claim 1, wherein the humidity measurements are given at relative humidity (RH) units and the method further comprises:
conducting a pressure calibration process comprising:
receiving second calibration humidity measurements from the humidity sensor, taken at various pressure levels, using a sample with known RH;
extracting at least one second calibration parameter from the second calibration RH measurements; and
determining a calibration pressure, based on the various pressure levels; and
calibrating the one or more third sets of RH measurements using the at least one second calibration parameter and the calibration pressure.

8. The method of claim 7, wherein the calibration pressure is selected from, an average pressure level of the various pressure levels, a median pressure level of the various pressure levels, a first pressure level from the various pressure levels, and a working pressure level at which the second gas sample is provided.

9. The method of claim 7, wherein the at least one second calibration parameter is a gradient of a linear relationship between the calibration RH measurements and the various pressure levels.

10. The method according to claim 1, further comprising:
displaying the calibrated one or more second sets of humidity measurements.

11. A method for increasing the accuracy of measurements of a humidity sensor, comprising:
receiving relative humidity (RH) measurements from a humidity sensor, taken at various temperatures, using a sample with known RH;
extracting at least one calibration parameter from the RH measurements;
determining a calibration temperature, based on the various temperatures; and
calibrating one or more additional sets of RH measurements using the at least one calibration parameter and the calibration temperature.

12. The method of claim 11, wherein the calibration temperature is selected from, an average temperature of the various temperatures, a median temperature of the various temperatures, a first temperature from the various temperatures, and a working temperature at which the second gas sample is provided.

13. The method of claim 11, wherein the at least one calibration parameter is a gradient of a linear relationship between the RH measurements and the various temperatures.

14. A method for increasing the accuracy of measurements from a humidity sensor, comprising:
receiving relative humidity (RH) measurements from a humidity sensor, taken at various pressure levels, using a sample with known RH;
extracting at least one calibration parameter from the RH measurements;
determining a calibration pressure, based on the various pressure levels; and
calibrating the one or more additional sets of RH measurements using the at least one calibration parameter and the calibration pressure.

15. The method of claim 14, wherein the calibration pressure is selected from, an average pressure level of the various pressure levels, a median pressure level of the various pressure levels, a first pressure level from the various pressure levels, and a working pressure level at which the second gas sample is provided.

16. The method of claim 14, wherein the at least one second calibration parameter is a gradient of a linear relationship between the calibration RH measurements and the various pressure levels.
receive one or more second sets of humidity measurements from the humidity sensor over a period of time, when the humidity sensor is exposed a to a second gas sample having an unknown humidity level; and
for each second set, subtracting the calibrating measurement from the humidity measurements.
